(19) United States
(12) Patent Application Publication
(10) Pub. No.: US 2002/0147422 A1
(43) Pub. Date: Oct. 10, 2002

(54) Custom-moldable patellar support, support product for improved patella tracking, and method

(76) Inventors: Thomas D. Darcey, Mooresville, NC (US); Kevin John O'Neill, Davidson, NC (US)

Correspondence Address: Joel Petrow, Smith & Nephew, Inc., 1450 Brooks Road, Memphis, TN, 38116, US

(21) Appl. No.: 09/779,726

(22) Filed: Feb. 8, 2001

(51) Int. Cl. A61F 13/00

(52) U.S. Cl. 602/62; 602/61

(57) ABSTRACT

A custom-moldable patellar support product for improved patella tracking in the femoral groove and relief of associated patella femoral pain syndrome, and including an outer container formed of moisture-impervious material and an initially-moldable, patellar support positioned in the container in substantially moisture-free conditions and sealed therein against entry of moisture until use. The support includes a substrate and a reactive system impregnated into or coated onto the substrate. The system remains stable when maintained in substantially moisture-free conditions and hardens upon exposure to moisture to form a rigid, self supporting structure. A flexible protective pad is positioned on one side of the substrate along its length to provide a cushioning barrier between the substrate and the skin of a patient when the patella support is in use. An outer cover covers the substrate on the side opposite the protective pad. The substrate, protective pad and outer cover are connected together into a unitary structure for being urged against the knee and into a position whereby the patella is repositioned into a therapeutic location relative to the femur for improved tracking in the femoral groove, and molded while flexible to the knee with the patella in the repositioned location. Several different types of retention devices, such as straps or bandages, may be used for retaining the patellar support on the knee in its molded location for maintaining the patella in the repositioned location.

### TECHNICAL FIELD AND BACKGROUND OF THE INVENTION

This invention relates to a custom-moldable patellar support, support product and method of improving patella tracking in the femoral groove and relieving associated patello-femoral pain syndrome.

The invention has particular application in the orthopedic medical field, where various knee injuries and diseases cause displacement of the patella, or knee cap, from the femoral groove, and thus pain in the knee area.

Over time, wear and tear underneath the patella can lead to degeneration of the cartilage behind the patella and cause pain, weakness and swelling of the knee joint. There are several different problems that affect the patella and the groove that it rides in as the knee is bent. The patella is wrapped inside a large tendon that connects the large muscles on the front of the thigh, the quadriceps muscles, to the lower leg bone. This large tendon when combined with the patella, is referred to as the quadriceps mechanism. The quadriceps mechanism allows the knee to be straightened. The patella acts as a fulcrum to increase the force of the quadriceps muscle. The underside of the patella is covered with articular cartilage, the smooth covering of joint surfaces. This slippery surface helps the patella glide in a special groove of the thigh bone, or femur.

Together the patella and the groove in the femur form the patello-femoral mechanism. Problems commonly develop when the patella suffers wear and tear. When the underlying cartilage begins to degenerate a condition referred to as chondromalacia patellae results. This wear and tear may develop for one or more reasons. Degeneration may develop as part of the aging process causing generalized wear and tear. The patello-femoral joint is usually affected as part of osteoarthritis of the knee. One of the more common causes of knee pain is a progressive change in the manner of movement of the patella through the patello-femoral groove as the knee moves. This problem, often referred to as a “tracking” problem, can result from a quadricep muscle imbalance. If part of the quadriceps muscles is weak for any reason a muscle imbalance can occur. When this happens, the pull of the quadriceps muscle on the patella may cause the patella to shift more to one side than the other. This causes uneven pressure on the articular cartilage on the side to which the patella is displaced.

Under normal circumstances, as the patella slides through the femoral groove, it shifts to the lateral aspect of the knee, thereby placing more pressure on one side of the underlying lateral cartilage. In time this pressure can cause damage to the articular cartilage.

Another type of imbalance may exist due to differences in how the bones of the knee are shaped. These differences, or anatomic variations, result in individuals who are born with a greater than normal angle where the femur and the tibia come together at the knee joint. Women tend to have a greater femoral/tibial angle than men, and can therefore be more prone to this condition. The patella normally sits at the center of this angle in the femoral groove. When the quadriceps muscle contracts, the force tries to straighten this angle, pushing the patella to the outside of the knee. In cases where this angle is increased, the patella tends to shift outward with greater pressure. This leads to a similar problem as above.

Finally, anatomic variations in the bones of the knee can occur where one side of the patello-femoral groove is smaller than normal. This creates a situation where the groove is too shallow, usually on the outside part of the knee. In individuals where the groove is too shallow, their patella may actually slip out of the groove, causing a patellar dislocation. This is not only painful when it occurs, but can damage the articular cartilage underneath the patella. If this occurs repeatedly, degeneration of the patello-femoral joint occurs fairly rapidly.

Chondromalacia patellae exists when there is damage to the articular cartilage underneath the patella. Many individuals experience vague pain in the knee which is difficult to localize. Pain may be felt along the inside edge of the patella. Typically, individuals who have patello-femoral problems experience pain when walking down stairs or down hills. Keeping the knee bent for long periods may also cause pain. The knee may feel as if it gives out on occasion. This is thought to be a reflex response to the pain and not because there is any instability in the knee. The knee may grind or a crunching sound may occur when squatting or when going up and down stairs. If there is a considerable amount of wear and tear, there may be a feeling of popping, or clicking, when the knee is bent. This results when the uneven surfaces of the underside of the patella and the femoral groove rub against one another. The knee may swell with heavy use and become stiff and tight. This is usually because of fluid accumulating inside the knee joint—sometimes called water on the knee. In the later stages, arthritis may be seen between the patella and femur.

Conventional treatments of these problems include decreasing the inflammation in the knee by rest and anti-inflammatory medications, such as aspirin, ibuprofen or more recent and exotic drugs such as sold under the trademarks Celebrex and Vioxx, sometimes in combination with physical therapy, particularly in the early stages.

Arthroscopic surgery is sometimes useful in the treatment of patello-femoral problems of the knee. If there are areas of articular cartilage damage behind the patella that are creating a rough surface, special tools can be used by the surgeon to smooth or shave the surface and thereby reduce pain. If the patella problems appear to be caused by malalignment, a procedure called “lateral release” may be employed to allow the patella to shift back to a more normal position and relieve pressure on the articular cartilage. The tight ligaments on the lateral side of the patella are cut, or released, to allow the patella to slide more towards the center of the femoral groove. These ligaments eventually heal with scar tissue that fills in the gap created by the surgery, but they no longer pull the patella to the outside as strong as before the surgery. This helps to balance the quadriceps mechanism and equalize the pressure on the articular cartilage behind the patella.

In some cases of severe malalignment, a lateral release alone may not be enough. If the malalignment is so bad that the patella dislocates repeatedly, then the operation will have to include a more involved re-alignment of the quadriceps mechanism. In addition to the lateral release, the tendons on the medial side may have to be tightened as well. In very severe cases of malaligmnent, the attachment of the patellar tendon may also have to be moved by moving the point where the patellar tendon attaches to the tibia, thereby changing the direction of movement of the patella through the patello-femoral groove. This is done surgically by removing the patellar tendon attachment with a piece of bone still attached to the tendon. The piece of bone with the tendon attached is then reattached to the tibia towards the medial side and allowed to heal. Usually, the bone is reattached using screws or a metal staple. Shifting the attachment of the patellar tendon medially also shifts the patella more medially. Once the surgery heals, the patella is expected to track more within the center of the patellar groove, spreading the pressure equally on the articular cartilage.

While all of these surgical procedures can successfully relieve pain in many instances, there does not presently exist a palliative treatment more extensive than rest and anti-inflammatory drugs, and less extensive than surgery.

The invention takes advantage of polymer chemistry to permit quick and easy molding of a patella support to the knee in such a manner as to gently force the patella back into a position where it tracks more properly in the femoral groove. The custom fit provides spreads contact between the support and the knee over a wider surface area, thereby reducing overall pressure while, at the same time, increasing pressure to the precise area necessary to shift the patella properly. This close, custom fit is in distinct contrast to “one size fits all” devices wherein a relatively thick cushioning pad is utilized to fill voids created by the distinctly irregular shape of the knee.

Therefore, in the particular embodiment of the invention disclosed in this application, the patella support will accommodate a wide range of knee sizes and can be fitted to either the right or left knee. Thus, a much reduced inventory of supports is required. This feature also substantially reduces design and manufacturing costs, and promotes use through ease of fitting. The custom-fit of the support permits easy removal for bathing, dressing or adjustment, and easy and mistake-proof replacement even by children.

The present invention permits quick and easy application of a support to the knee in such a way as to achieve a true custom fit. The moisture curable resin system used results in a very rigid patella support which permanently holds the shape of the knee to which it is molded. No heat is required, and a source of water is the only additional material necessary to achieve a cure. Atmospheric moisture alone will cure the support into its hardened position in a relatively short period of time, but in practice the resin in or on the support will typically be activated by dipping in water and then removing the excess by rolling the splint in a towel immediately before application.

### SUMMARY OF THE INVENTION

Therefore, it is an object of the invention to provide a custom-moldable patellar support.

It is another object of the invention to provide a patellar support which can be molded to a knee to stabilize the patella in a displaced position whereby the patella more properly tracks in the femoral groove.

It is another object of the invention to provide patella support which can be custom-molded to a particular patient.

It is another object of the invention to provide patellar support having a shape prior to forming which permits the support to be formed onto either the left or the right knee.

It is another object of the invention to provide a patellar support which hardens in the presence of moisture to form a very rigid but very lightweight support.

It is another object of the invention to provide a patellar support which is stored in a moisture-proof pouch until ready for application to the knee.

It is another object of the invention to provide a patellar support which is custom-molded to a patient's knee in such a way that it can be initially held in place with an elastic bandage to reduce edema on the knee, and thereafter held in place with straps during to maintain the patellar in its displaced location.

These and other objects of the present invention are achieved in the preferred embodiments disclosed below by providing a custom-moldable patellar support product for improved patella tracking in the femoral groove and relief of associated patella femoral pain syndrome, and comprising an outer container formed of moisture-impervious material and an initially-moldable, arcuate patellar support positioned in the container in substantially moisture-free conditions and sealed therein against entry of moisture until use. The support comprises a substrate and a reactive system impregnated into or coated onto the substrate. The system remains stable when maintained in substantially moisture-free conditions and hardens upon exposure to moisture to form a rigid, self supporting structure. A flexible protective pad is positioned on one side of the substrate along its length to provide a cushioning barrier between the substrate and the skin of a patient when the patella support is in use. An outer cover covers the substrate on the side opposite the protective pad. The substrate, protective pad and outer cover are connected together into a unitary structure for being urged against the knee and into a position whereby the patella is repositioned into a therapeutic location relative to the femur for improved tracking in the femoral groove, and molded while flexible to the knee with the patella in the repositioned location. Retention means may be provided for retaining the patellar support on the knee in its molded location for maintaining the patella in the repositioned location.

According to one preferred embodiment of the invention, the container is fabricated of an aluminum foil laminate having an outer tear resistant layer, a central aluminum foil layer and an inner heat sealable plastic layer.

According to another preferred embodiment of the invention, the support is circular.

According to yet another preferred embodiment of the invention, the circular support includes a centrally-disposed patellar opening.

According to yet another preferred embodiment of the invention, the support includes at least one radially-extending hinge formed in the support for permitting diametrical flexure of the support during molding to the knee and during use of the hardened support on the knee.

According to yet another preferred embodiment of the invention, a pair of aligned, opposed and radially-extending hinges is formed in the support for permitting diametrical flexure during molding to the knee and during use of the hardened support on the knee.

According to yet another preferred embodiment of the invention, the pair of hinges bisect the support.

According to yet another preferred embodiment of the invention, the arcuate support is C-shaped and thereby includes a centrally-disposed patellar opening.

According to yet another preferred embodiment of the invention, the support includes at least one radially-extending hinge formed in the support for permitting diametrical flexure of the support during molding to the knee and during use of the hardened support on the knee.

Preferably, the hinge bisects the support.

According to yet another preferred embodiment of the invention, a custom-moldable patellar support for improved patella tracking in the femoral groove and relief of associated patella femoral pain syndrome is provided, comprises an initially-moldable, arcuate patellar support for being initially positioned in a container in substantially moisture-free conditions and sealed therein against entry of moisture until use. The support comprises a substrate and a reactive system impregnated into or coated onto the substrate. The system remains stable when maintained in substantially moisture-free conditions and hardening upon exposure to moisture to form a rigid, self supporting structure. A flexible protective pad is positioned on one side of the substrate along its length to provide a cushioning barrier between the substrate and the skin of a patient when the patella support is in use. An outer cover covers the substrate on the side opposite the protective pad, and the substrate, protective pad and outer cover are connected together into a unitary structure for being urged against the knee and into a position whereby the patella is repositioned into a therapeutic location relative to the femur for improved tracking in the femoral groove, and molded while flexible to the knee with the patella in the repositioned location for thereafter being retained on the knee in its molded location for maintaining the patella in the repositioned location.

An embodiment of the method of improving patella tracking in the femoral groove and relieving associated patella femoral pain syndrome according to the invention comprises the steps of providing an initially-moldable, arcuate patellar support positioned in the container in substantially moisture-free conditions and sealed therein against entry of moisture until use, the support comprising, a substrate, a reactive system impregnated into or coated onto the substrate, the system remaining stable when maintained in substantially moisture-free conditions and hardening upon exposure to moisture to form a rigid, self supporting structure, a flexible protective pad positioned on one side of the substrate along its length to provide a cushioning barrier between the substrate and the skin of a patient when the patella support is in use and an outer cover covering the substrate on the side opposite the protective pad, the substrate, protective pad and outer cover connected together into a unitary structure. The support is wetted with water. While still flexible, the support is urged against the knee and into a position whereby the patella is repositioned into a therapeutic location relative to the femur for improved tracking in the femoral groove. The support while still flexible is molded to the knee with the patella in the repositioned location and allowed to harden on the knee with the patella in the repositioned location.

According to one preferred embodiment of the invention, the method includes the step of retaining the patellar support on the knee in its molded location for maintaining the patella in the repositioned location.

According to another preferred embodiment of the invention, the method includes the step of hinging the support to permit more effective molding of the support to the knee.

According to yet another preferred embodiment of the invention, the method includes the step of providing a centrally-disposed opening in the support for being positioned directly over the patella.

### BRIEF DESCRIPTION OF THE DRAWINGS

Some of the objects of the invention have been set forth above. Other objects and advantages of the invention will appear as the description proceeds when taken in conjunction with the following drawings, in which:

FIG. 1 is a simplified front elevation of the muscles, tendons, ligaments and bones of a human knee;

FIG. 2 is a simplified side elevation according to FIG. 1;

FIG. 3 is a view of a normal patella residing properly in the femoral groove;

FIG. 4 is a view of a diseased patella laterally-offset from the femoral groove;

FIG. 5 is a view of the diseased patella shown in FIG. 2 displaced into a position to achieve more proper tracking in the femoral groove by an embodiment of the invention;

FIG. 6 is a plan view of a patellar support according to one embodiment of the invention;

FIG. 7 is a fragmentary plan view of the embodiment in FIG. 6 with layers removed to show the sandwich-like construction of the patellar support;

FIG. 8 is a perspective view of the patellar support product according to an embodiment of the invention;

FIG. 9 shows removal of the patellar support from the container of the patellar support product immediately prior to use;

FIG. 10 illustrates one manner of wetting the patellar support with water immediately prior to application to the knee;

FIG. 11 is a view of the patellar support according to the embodiment of the invention shown in FIGS. 6-10 in place on a knee;

FIG. 12 is a view of a patellar support according to a modified embodiment of the patellar support shown in FIGS. 6-10 in place on a knee;

FIG. 13 is a view of a patellar support according to a further modified embodiment of the patellar support in place on a knee;

FIG. 14 is a view of a patellar support according to a further modified embodiment of the patellar support;

FIG. 15 is a view of the patellar support according to FIG. 14 in place on a knee; and

FIG. 16 is a view of a patellar support according to a further modified embodiment of the patellar support.

### DESCRIPTION OF THE PREFERRED EMBODIMENT AND BEST MODE

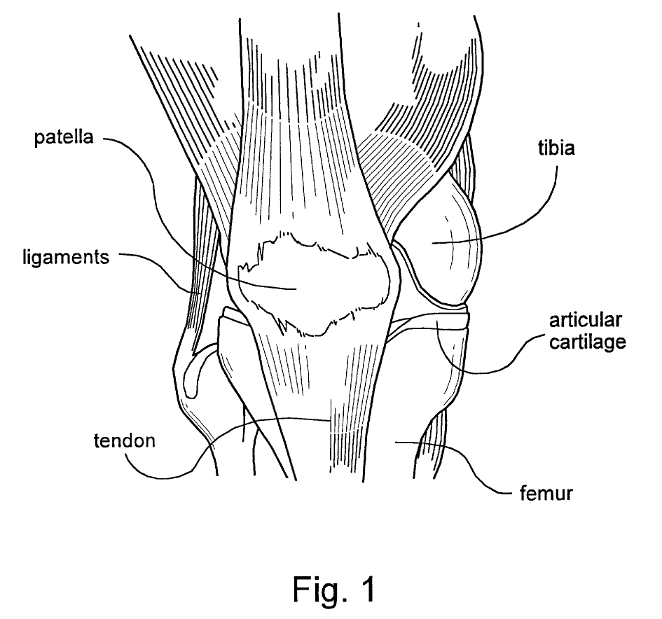

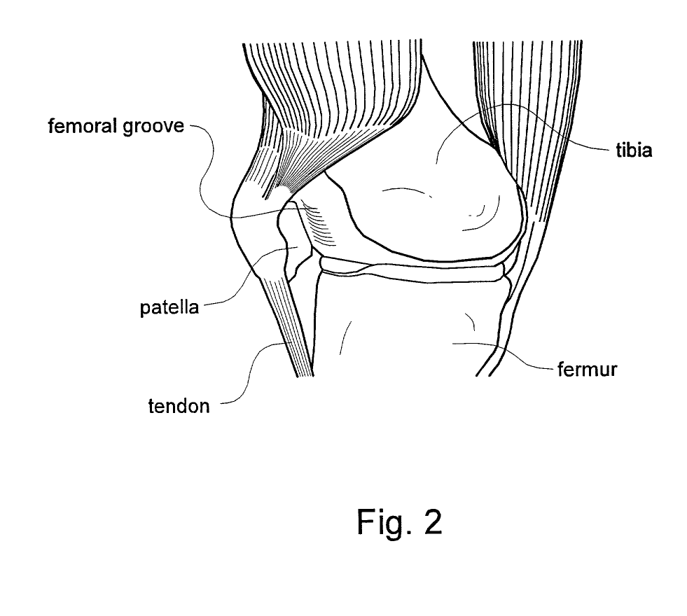

Referring to FIGS. 1-5, anatomy of the knee sufficient to explain the invention is shown. As is shown in FIGS. 1 and 2, the patella is wrapped inside a large tendon that connects the large muscles on the front of the thigh, the quadriceps muscles, to the lower leg bone, the femur. This quadriceps mechanism allows the knee to be straightened. The patella acts as a fulcrum to increase the force of the quadriceps muscle. The underside of the patella is covered with articular cartilage, the smooth covering of joint surfaces. This slippery surface helps the patella glide in the femoral groove of the femur. See FIG. 2.

The patella and the groove in the femur form the patello-femoral mechanism, and can deteriorate as a result of injury, wear or disease. When the underlying articular cartilage begins to degenerate, chondromalacia patellae results.

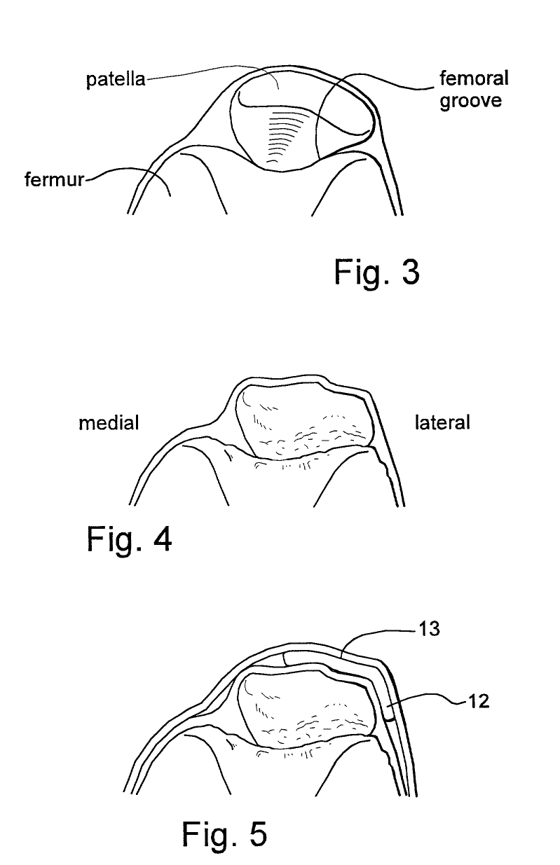

As is shown in FIG. 3, in a normal, healthy knee the posterior aspect of the patella resides in the femoral groove and glides up and down as the knee is articulated. The posterior surface of the patella and the adjacent anterior surface of the femoral joint defining the femoral groove are smooth and even as a result of the covering of articular cartilage.

A typical case of chondromalacia patellae is shown in FIG. 4, and, as can be seen, the adjacent surfaces of the patella and femoral groove are irregular, pitted and deformed. The decreased surface area of contact increases pressure and wear on the remaining areas of contact, thus accelerating further degeneration of the contacting surfaces. The patella has shifted to lateral side of the knee. This condition can cause pain, swelling, inflammation and thus reduced mobility. Progressive deterioration can eventually require surgery, including replacement of the effected parts.

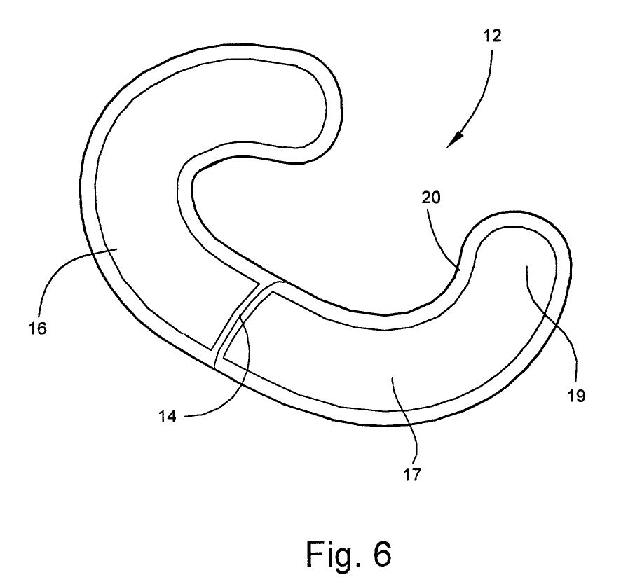

The basic principle of the invention is illustrated in FIG. 5. One preferred embodiment of a patellar support, such as the patellar support 12 shown in FIG. 6, is positioned on the knee in such a position as to at least partially return the patella to a more centrally-oriented position in the femoral groove, understanding, of course, that the underlying deterioration of the joint remains. With the patellar support 12 in a position whereby the patella tracks along the femoral groove, the patellar support 12 is allowed to harden, as described below, so that it remains in a shape which conforms to the part of the knee to which it was placed, and where it displaces the patella back to a more normal position. The patellar support 10 is held in position with one or more straps 13, or an elastic bandage, tube or hose. Pain is reduced, mobility increased and further deterioration slowed. In contrast to thick pads which must be conformed to the knee by significant pressure, the precisely molded shape of the invention permits pressure to be uniformly distributed across the surface area of the patellar support 12, thus reducing pressure on the already abnormal knee joint. The straps 13 or other retention means merely exert enough pressure to maintain the patellar support 12 in the correct position to maintain the displacement of the patella.

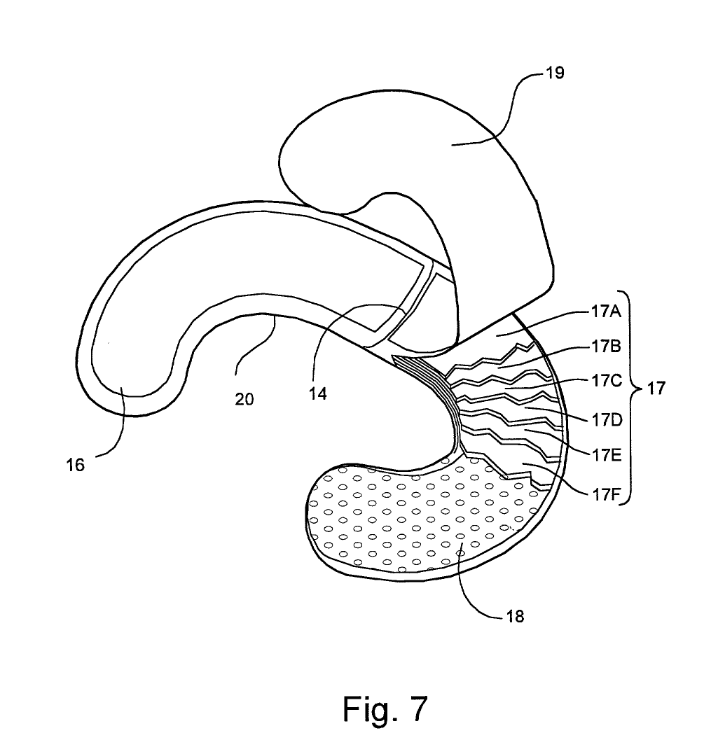

Referring now to FIGS. 6 and 7, the patellar support 12 according to one preferred embodiment of the invention is illustrated broadly and is a generally arcuate C-shaped structure sized to partially encircle the patella. The patellar support 12 can be oriented as needed to achieve the desired displacement of the patella. The particular structure of the patellar support 12 includes a centrally-disposed and radially-extending hinge 14 formed by sewing stitches. As shown in phantom lines, this particular embodiment includes two individual substrate elements 16 and 17, with the hinge formed between them, so that the patellar support 12 will flex along the hinge after molding and curing, as described below.

As used herein, the term “arcuate” is used to describe a shape whereby the longitudinal axis of the support is displaced from a straight line sufficiently to extend around a predetermined range of arc of the patella, and not whether the shape is smoothly curved or formed by a series of adjacent straight line segments.

This structure is more specifically shown in FIG. 7. Substrate element 17 is formed of, for example, six layers of woven fiberglass fabric 17A-F overlaid in registration with each other to form a laminated structure. Substrate element 16 is identically formed, and the disclosure below is equally applicable to both substrate elements 16 and 17. Other fabric material and constructions, such as knitted polypropylene, can also be used for the substrate fabric.

The fiberglass or other fabric layers 17A-F of the substrate 17 are impregnated or coated with a moisture-curable resin such as polyisocyanate as described in full in the present applicant's U.S. Pat. No. 4,770,299. This reactive system remains stable when maintained in substantially moisture-free conditions, such as in a moisture-impervious pouch, but hardens upon exposure to sufficient moisture to form a rigid, self-supporting structure. A typical formulation of the reactive system is set forth in the following table:

Typical Formulation:

| | | |
|---|---|---|
| Isonate ↓ 143 L or | polyisocyanate | 50.0% |
| Mondur ↓ CD or | | |
| Rubinate ↓ X1168 | | |
| Pluracol ↓ P1010 | polyol | 46.6% |
| DC-200 Silicone | defoaming agent | 0.30% |
| Benzoyl Chloride | stabilizer | 0.10% |
| Thancat ↓ DM-70 | catalyst | 3.0% |
| | | 100% |

A complete discussion of the parameters of the reactive system, the manner of production and the variables which apply are found in U.S. Pat. No. 4,411,262.

The polyisocyanate resin remains in a viscous, liquid unhardened state so long as the resin is not exposed to moisture. This permits the substrate elements 16 and 17 to remain flexible and moldable so long as the resin is not exposed to moisture, and for a relatively short period of time after exposure to moisture. The curing time can be controlled to some extent by the quantity and temperature of the water to which the resin is exposed. For example, exposure to water by dipping will result in quite rapid curing, while merely allowing the resin to be exposed to air will cause long curing times proportional to the amount of moisture in the air to which it is exposed.

The resin coated or impregnated substrate elements 16 and 17 are covered with a foam protective pad 18, which may be a single thickness or a laminated structure. One preferred embodiment is a 3/16 inch, six pound EVA (ethylene vinyl acetate) pad. Another embodiment may be a ⅜ inch laminated pad of a ⅛ inch outer EVA pad and a ¼ inch outer polyethylene/polyurethane, combination open and closed cell foam. Spaced-apart ventilation holes 18A permit rapid penetration of water to the substrate 16 during wetting and curing, and permit improved air flow and cooling while being worn by the patient.

The pad 18 covers and provides cushioning between the skin and the rigid substrate elements 16 and 17. The pad 18 is flexible enough to bend easily with the other components of the patellar support 12 during fitting and curing. The pad 18 extends the entire length of the patellar support 12.

A fabric outer cover 19 such as a woven polyester fabric, covers the side of the substrate elements 16 and 17 opposite the side covered by the foam pad 18. The fabric cover 19 joined to the foam pad by, for example, an overedge or serging seam 20 directly to the edges of the foam pad 19 enclosing the substrate elements 16 and 17.

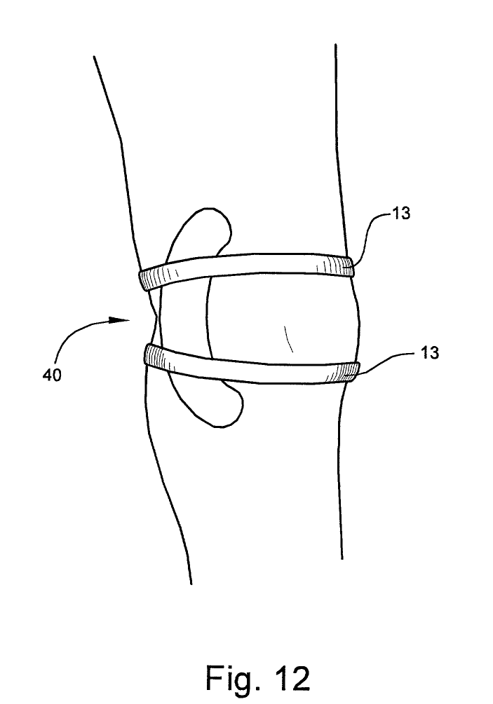

The hinge 14 is clearly optional. In many applications a patellar support substantially the same overall shape as patellar support 12 but without the hinge and with a single substrate element will suffice. See FIG. 12.

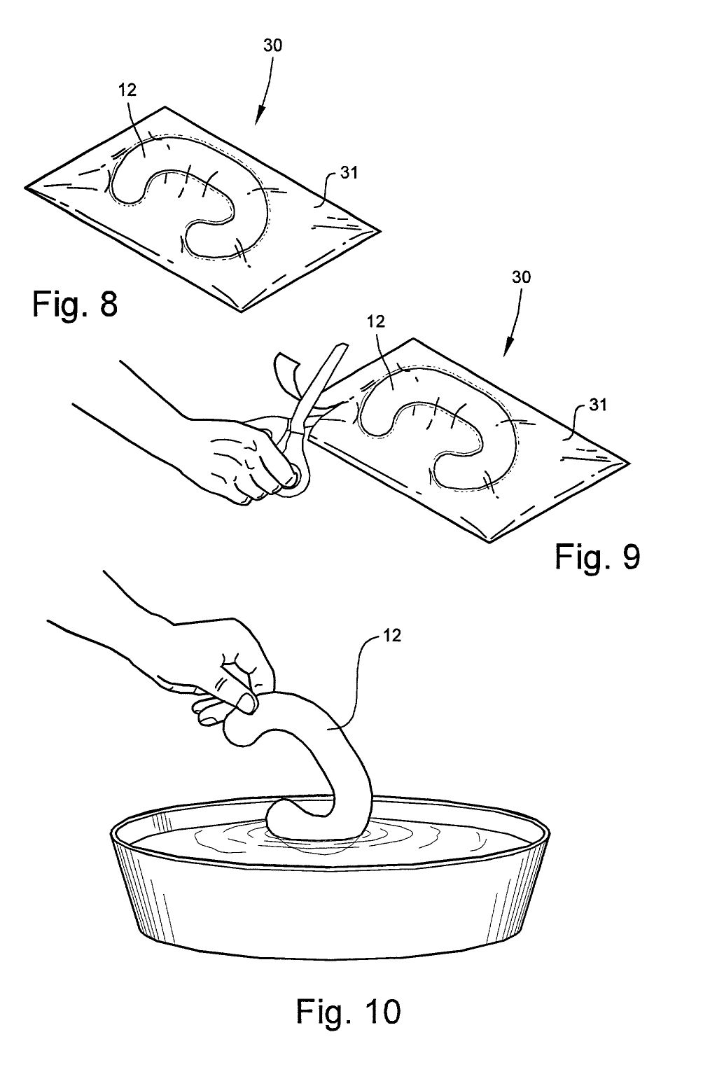

The patellar support product 30 of which the patellar support 12 is a part is shown in FIG. 8. A sealed, moisture-impervious foil and plastic laminated pouch or container 31 is fabricated of an aluminum foil laminate having an outer tear resistant layer, a central aluminum foil layer and an inner heat sealable plastic layer. Container 31 is opened with scissors or a knife, see FIG. 9, and the patellar support 12 according to the above-described embodiment of the invention is removed.

As is shown in FIG. 10, the patellar support 12 which has just been removed from the protective container 31 is dipped in water to activate the moisture-curable resin described above.

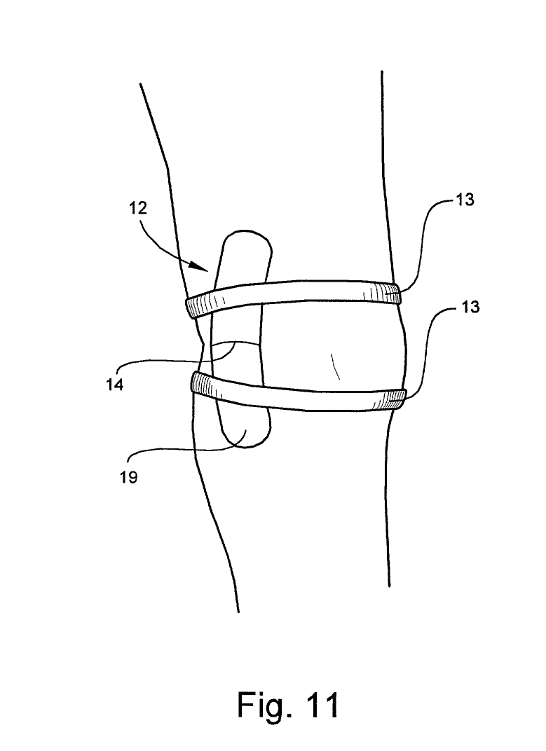

As shown in FIG. 11, the wetted and still flexible patellar support 12 is then placed on the knee and pressure is applied as it is moved around on the knee until the physician determines that the patella as been displaced from its original location back to a more normal position, as shown above in FIG. 5. The patellar support 12 can be rotated on the knee about a point defined by the patella as necessary to move the patella medially, laterally, upwardly or downwardly. When the proper position has been obtained, temporary retention means, such as the straps 13, are applied and the patellar support 12 is allowed to cure. Thereafter, it is necessary only to replace the patellar support 12 in the same location and retain in place with the straps 13. Correct positioning is facilitated by the permanently molded configuration by which it can be easily determined that the correct position has been resumed. The patellar support 12 is usable on either the right or left knee. With no voids to fill or accommodate as the patient moves about, the pressure exerted by the patellar support 12 is very evenly spread over the maximum practical surface area of the knee, thereby reducing the possibility of chafing or rubbing at points of uneven pressure. As is shown in FIGS. 11 and 12, a properly applied patellar support 13 resides generally on either the medial or lateral side of the patella.

Also shown in FIG. 12 is a patellar support 40 which is constructed and used as described above with reference to patellar support 12, except that the bisecting hinge 14 is omitted. The substrate may thus be a single, unitary structure of laminated fabric layers, instead of being divided into separate substrate structures on either side of the hinge. In another alternative, the hinge can be formed merely by providing two spaced-apart substrate structures, with the hinge being the space between the substrate structures without any sewing stitches.

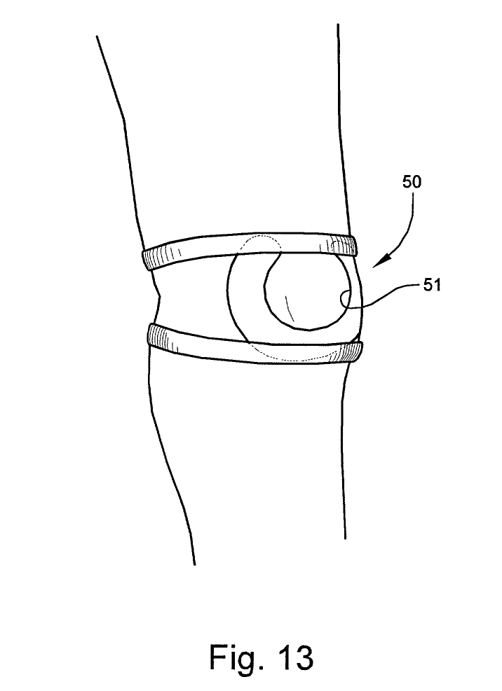

Another embodiment is shown in FIG. 13. A horseshoe-shaped patellar support 50 is positioned around approximately three-fourths of the circumference of the patella and is held in place by straps 13. The patellar support 50 includes a centrally-disposed patellar opening 51 defined by the inner peripheral edge of the patellar support 50. As described above, the patellar support 50 can be rotated and positioned optimally to displace the patella into a position where it will more properly track in the femoral groove. The patellar support 50 is constructed of the same components as described above with reference to the patellar support 12, and used in the same manner, the moisture-curable resin substrate permitting placement in a patella-displacing position while flexible.

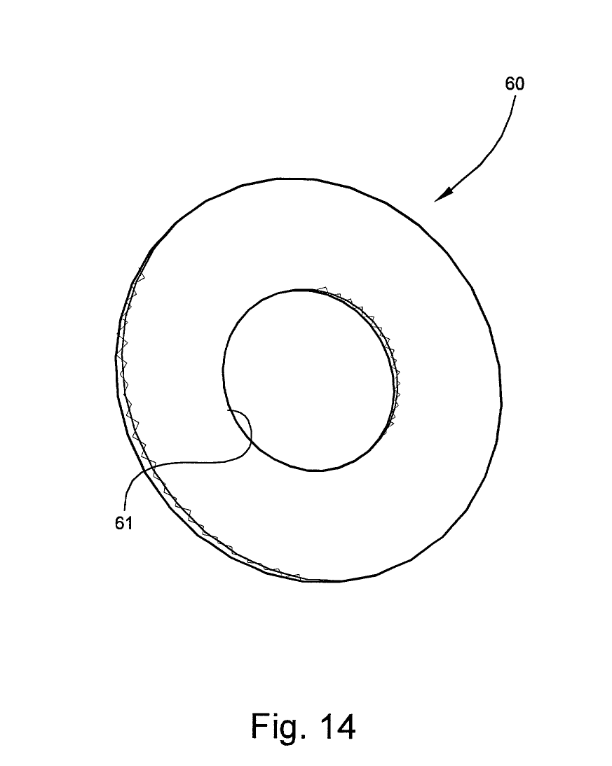

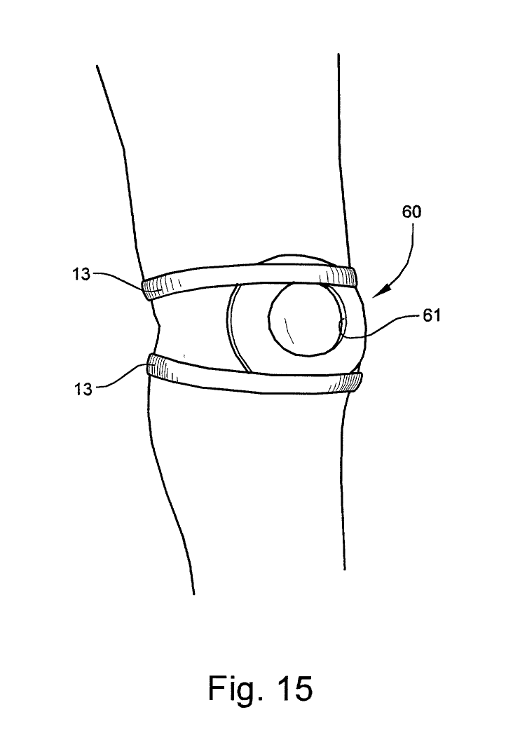

Another embodiment of the invention is shown in FIGS. 14 and 15. A circular patellar support 60 is constructed to completely encircle the circumference of the patella and be held in place by straps 13. The patellar support 60 includes a centrally-disposed patellar opening 61 defined by the inner peripheral edge of the patellar support 60. As described above, the patellar support 60 can be rotated and positioned optimally to displace the patella into a position where it will more properly track in the femoral groove. The patellar support 60 is constructed of the same components as described above with reference to the patellar support 12, and used in the same manner, the moisture-curable resin substrate permitting placement in a patella-displacing position while still flexible.

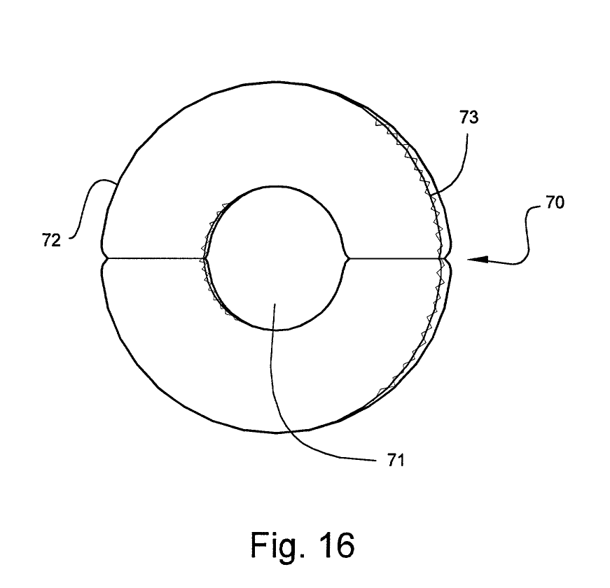

Another modified embodiment of the patellar support 60 is shown in FIG. 16 at reference numeral 70. The circular patellar support 70 is constructed to completely encircle the circumference of the patella and be held in place by straps 13 and includes a centrally-disposed patellar opening 71 defined by the inner peripheral edge of the patellar support 70. As described above, the patellar support 70 can be rotated and positioned optimally to displace the patella into a position where it will more properly track in the femoral groove. The patellar support 70 is constructed of the same components as described above with reference to the patellar support 12, and used in the same manner, the moisture-curable resin substrate permitting placement in a patella-displacing position while flexible.

A pair of aligned, opposed and radially-extending hinges 72, 73 are formed by sewing stitches in the support for permitting diametrical flexure during molding to the knee and during use of the hardened support on the knee. As with the patellar support 12, the patellar support 70 has a substrate which is divided into two separate but identical substrate elements (not shown) which preferably have the same laminate structure shown in FIG. 7, but are semi-circularly shaped.

Other means of securing the patellar support into a unit and forming the hinges may be used, for example, adhesives, ultrasonic welding or thermoplastic heat fusing, as appropriate to the materials used.

A patellar support, patellar support product and method is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

We claim:

1. A custom-moldable patellar support product for improved patella tracking in the femoral groove and relief of associated patella femoral pain syndrome, comprising:
(a) an outer container formed of moisture-impervious material;
(b) an initially-moldable, arcuate patellar support positioned in the container in substantially moisture-free conditions and sealed therein against entry of moisture until use, and comprising:
(i) a substrate;
(ii) a reactive system impregnated into or coated onto the substrate, the system remaining stable when maintained in substantially moisture-free conditions and hardening upon exposure to moisture to form a rigid, self supporting structure;
(iii) a flexible protective pad positioned on one side of the substrate along its length to provide a cushioning barrier between the substrate and the skin of a patient when the patella support is in use;
(iv) an outer cover covering the substrate on the side opposite the protective pad;
(v) said substrate, protective pad and outer cover connected together into a unitary structure for being urged against the knee and into a position whereby the patella is repositioned into a therapeutic location relative to the femur for improved tracking in the femoral groove, and molded while flexible to the knee with the patella in the repositioned location; and
(b) retention means for retaining the patellar support on the knee in its molded location for maintaining the patella in the repositioned location.

2. A patellar support product according to claim 1, wherein the container is fabricated of an aluminum foil laminate having an outer tear resistant layer, a central aluminum foil layer and an inner heat sealable plastic layer.

3. A patellar support product according to claim 1, wherein said support is circular.

4. A patellar support product according to claim 3, wherein said circular support includes a centrally-disposed patellar opening.

5. A patellar support product according to claim 3 or 4, and including at least one radially-extending hinge formed in said support for permitting diametrical flexure of the support during molding to the knee and during use of the hardened support on the knee.

6. A patellar support product according to claim 4, and including a pair of aligned, opposed and radially-extending hinges formed in said support for permitting diametrical flexure during molding to the knee and during use of the hardened support on the knee.

7. A patellar support product according to claim 6, wherein said pair of hinges bisect the support.

8. A patellar support product according to claim 1, wherein said arcuate support is C-shaped and thereby includes a centrally-disposed patellar opening.

9. A patellar support product according to claim 8, and including at least one radially-extending hinge formed in said support for permitting diametrical flexure of the support during molding to the knee and during use of the hardened support on the knee.

10. A patellar support product according to claim 9, wherein said hinge bisects the support.

11. A custom-moldable patellar support for improved patella tracking in the femoral groove and relief of associated patella femoral pain syndrome, comprising:
(a) an initially-moldable, arcuate patellar support for being initially positioned in a container in substantially moisture-free conditions and sealed therein against entry of moisture until use, and comprising:
(i) a substrate;
(ii) a reactive system impregnated into or coated onto the substrate, the system remaining stable when maintained in substantially moisture-free conditions and hardening upon exposure to moisture to form a rigid, self supporting structure;
(iii) a flexible protective pad positioned on one side of the substrate along its length to provide a cushioning barrier between the substrate and the skin of a patient when the patella support is in use;
(iv) an outer cover covering the substrate on the side opposite the protective pad;
(v) said substrate, protective pad and outer cover connected together into a unitary structure for being urged against the knee and into a position whereby the patella is repositioned into a therapeutic location relative to the femur for improved tracking in the femoral groove, and molded while flexible to the knee with the patella in the repositioned location for thereafter being retained on the knee in its molded location for maintaining the patella in the repositioned location.

12. A patellar support according to claim 11, wherein the container is fabricated of an aluminum foil laminate having an outer tear resistant layer, a central aluminum foil layer and an inner heat sealable plastic layer.

13. A patellar support product according to claim 11, wherein said support is circular.

14. A patellar support product according to claim 13, wherein said circular support includes a centrally-disposed patellar opening.

15. A patellar support product according to claim 13 or 14, and including at least one radially-extending hinge formed in said support for permitting diametrical flexure of the support during molding to the knee and during use of the hardened support on the knee.

16. A patellar support product according to claim 14, and including a pair of aligned, opposed and radially-extending hinges formed in said support for permitting diametrical flexure during molding to the knee and during use of the hardened support on the knee.

17. A patellar support product according to claim 16, wherein said pair of hinges bisect the support.

18. A patellar support product according to claim 11, wherein said arcuate support is C-shaped and thereby includes a centrally-disposed patellar opening.

19. A patellar support product according to claim 18, and including at least one radially-extending hinge formed in said support for permitting diametrical flexure of the support during molding to the knee and during use of the hardened support on the knee.

20. A patellar support product according to claim 19, wherein said hinge bisects the support.

21. A patellar support according to claim 1 or 11, wherein the substrate comprises a plurality of knitted, woven or non-woven fabric layers.

22. A patellar support according to claim 1 or 11, wherein the protective pad comprises a foam material.

23. A patellar support according to claim 22, wherein the foam material is chosen from the group consisting of open or closed cell EVA or polyurethane.

24. A patellar support according to claim 1 or 11, wherein the outer cover is formed of a synthetic, hydrophobic fabric.

25. A patellar support according to claim 1 or 11, wherein the reactive system comprises a blended polyisocyanate, polyol, catalyst and stabilizer.

26. A patellar support according to claim 1 or 11, wherein said protective padding, substrate and outer cover are sandwiched together in overlying layers and joined together around their respective peripheral edges by sewing stitches to form a unitary structure.

27. A method of improving patella tracking in the femoral groove and relieving associated patella femoral pain syndrome, comprising the steps of:
(a) providing an initially-moldable, arcuate patellar support positioned in the container in substantially moisture-free conditions and sealed therein against entry of moisture until use, said support comprising:
(i) a substrate;
(ii) a reactive system impregnated into or coated onto the substrate, the system remaining stable when maintained in substantially moisture-free conditions and hardening upon exposure to moisture to form a rigid, self supporting structure;
(iii) a flexible protective pad positioned on one side of the substrate along its length to provide a cushioning barrier between the substrate and the skin of a patient when the patella support is in use;
(iv) an outer cover covering the substrate on the side opposite the protective pad, said substrate, protective pad and outer cover connected together into a unitary structure;
(b) wetting said support;
(c) urging said support against the knee and into a position whereby the patella is repositioned into a therapeutic location relative to the femur for improved tracking in the femoral groove;
(d) molding the support while flexible to the knee with the patella in the repositioned location; and
(e) allowing the support to harden on the knee with the patella in the repositioned location.

28. A method according to claim 27, and including the step of retaining the patellar support on the knee in its molded location for maintaining the patella in the repositioned location.

29. A method according to claim 28, and including the step of hinging the support to permit more effective molding of the support to the knee.

30. A method according to claim 28, and including the step of providing a centrally-disposed opening in the support for being positioned directly over the patella.
